(19) United States
(12) Patent Application Publication
(10) Pub. No.: US 2026/0345291 A1
(43) Pub. Date: Nov. 5, 2026

(54) FIDUCIAL MARKER ASSEMBLIES FOR SURGICAL NAVIGATION SYSTEMS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Kevin Santos Nunes, Coimbra (PT); Luis Carlos Fial Teixeira Ribeiro, Coimbra (PT); Rui Jorge Melo Teixeira, Tondela (PT); João Pedro de Almeida Barreto, Coimbra (PT); Michel Goncalves Almeida Antunes, Coimbra (PT)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/487,587

(22) PCT Filed: Jun. 7, 2024
(86) PCT No.: PCT/US2024/032963

§ 371 Date: Nov. 25, 2025

Related U.S. Application Data
(60) Provisional application No. 63/471,897, filed on Jun. 8, 2023.

(51) Int. Cl. A61B 90/96; A61B 34/20; A61B 90/00

(52) U.S. Cl. CPC A61B 90/96; A61B 34/20; A61B 2034/2055; A61B 2090/3916; A61B 2090/3937; A61B 2090/3991

(57) ABSTRACT

A fiducial marker for use with a surgical navigation system includes a body portion, a top portion, and a plurality of faces arranged on the body portion and the top portion. Each of the plurality of faces includes a fiducial, and the plurality of faces is arranged such that at least two of the fiducials are visible to the surgical navigation system in substantially any orientation of the fiducial marker within a surgical environment. In an example, the body portion is hexagonal and the top portion is triangular. In another example, first and second channels arranged on a bottom-facing side of the fiducial marker.

### CROSS-REFERENCE TO RELATED APPLICATIONS

The present application claims priority to U.S. Provisional Patent Application No. 63/471,897, titled FIDUCIAL MARKER ASSEMBLIES FOR SURGICAL NAVIGATION SYSTEMS, filed Jun. 8, 2023, which is hereby incorporated by reference herein in its entirety.

### TECHNICAL FIELD

The present disclosure relates generally to methods, systems, and apparatuses related to a computer-assisted surgical system that includes various hardware and software components that work together to enhance surgical workflows. The disclosed techniques may be applied to, for example, shoulder, hip, and knee arthroplasties, as well as other surgical interventions such as arthroscopic procedures, spinal procedures, maxillofacial procedures, neuro-surgery procedures, rotator cuff procedures, ligament repair and replacement procedures.

### BACKGROUND

Many modern orthopedic surgical procedures are performed with assistance from a surgical navigation system. For example, total knee arthroplasty (TKA) is an orthopedic procedure that involves the replacement of damaged knee articular surfaces (femoral condyles and tibial plateau) with metal and polyethylene components for patients that generally have severe arthritis or a severe knee injury. To place those components, the surgeon needs to perform precise bone resections to obtain the best positioning of the implant using a complete set of standard instrumentation available for all different shapes and sizes of knees.

The problem is that the surgeon needs to verify which instruments best fit on each patient intraoperatively and use those standard instruments to perform the bone resections, which can lead to those cuts not being as precise as they should be. Furthermore, conventional TKA instrumentation is composed of instruments of multiple sizes, usually delivered in multiple instrumentation trays, which represent a significant capital equipment cost for the manufacturer and sterilization cost for the healthcare facility.

### SUMMARY

The present disclosure is directed to fiducial marker assemblies for surgical navigation systems.

In one embodiment, the present disclosure is directed to a fiducial marker for use with a surgical navigation system, the fiducial marker comprising: a plurality of faces, each of the plurality of faces comprising a fiducial configured to be identified by the surgical navigation system; wherein at least two of the fiducials are configured to be visible to the surgical navigation system from substantially any orientation; and a connector configured to engage with a probe.

In one embodiment, the present disclosure is directed to a fiducial marker for use with a surgical navigation system, the fiducial marker comprising: a plurality of faces, each of the plurality of faces comprising a fiducial configured to be identified by the surgical navigation system; wherein at least two of the fiducials are configured to be visible to the surgical navigation system from substantially any orientation; and a channel configured to receive a fastener therethrough; wherein the fiducial marker is configured to not rotate with respect to the fastener when the fastener is inserted through the channel.

In one embodiment, the present disclosure is directed to a surgical equipment kit for use with a surgical navigation system, the surgical equipment kit comprising: a probe configured to be received by one or more pieces of other surgical equipment; and a fiducial marker comprising: a plurality of faces, each of the plurality of faces comprising a fiducial configured to be identified by the surgical navigation system, wherein at least two of the fiducials are configured to be visible to the surgical navigation system from substantially any orientation, and a connector configured to engage with the probe.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical data server via a network in accordance with an embodiment.

FIG. 3A depicts a perspective view of a fiducial marker configured to be affixed to a bone in accordance with an embodiment of the present disclosure.

FIG. 3B depicts a reverse perspective view of the fiducial marker of FIG. 3A in accordance with an embodiment of the present disclosure.

FIG. 3C depicts an overhead view of the fiducial marker of FIG. 3A in accordance with an embodiment of the present disclosure.

FIG. 3D depicts a perspective view of a first step of securing fasteners to the fiducial marker of FIGS. 3A-3C in accordance with an embodiment of the present disclosure.

FIG. 3E depicts a perspective view of a second step of securing fasteners to the fiducial marker of FIGS. 3A-3C in accordance with an embodiment of the present disclosure.

FIG. 3F depicts a perspective view of a third step of securing fasteners to the fiducial marker of FIGS. 3A-3C in accordance with an embodiment of the present disclosure.

FIG. 3G depicts a perspective view of a fourth step of securing fasteners to the fiducial marker of FIGS. 3A-3C in accordance with an embodiment of the present disclosure.

FIG. 3H depicts a perspective view of a first step of securing the fiducial marker of FIGS. 3A-3C to a bone using two fasteners in accordance with an embodiment of the present disclosure.

FIG. 3I depicts a perspective view of a second step of securing the fiducial marker of FIGS. 3A-3C to a bone using two fasteners in accordance with an embodiment of the present disclosure.

FIG. 3J depicts a perspective view of a third step of securing the fiducial marker of FIGS. 3A-3C to a bone using two fasteners in accordance with an embodiment of the present disclosure.

FIG. 3K depicts a perspective view demonstrating that the fiducial marker is secured from both translation and rotation movement once secured using two fasteners as shown in FIGS. 3H-3J in accordance with an embodiment of the present disclosure.

FIG. 4A depicts a perspective view of a second embodiment of a fiducial marker configured to be affixed to a bone in accordance with an embodiment of the present disclosure.

FIG. 4B depicts an overhead view of the fiducial marker of FIG. 4A in accordance with an embodiment of the present disclosure.

FIG. 4C depicts a perspective view of a first step securing fasteners to the fiducial marker of FIGS. 4A and 4B in accordance with an embodiment of the present disclosure.

FIG. 4D depicts a perspective view of a second step securing fasteners to the fiducial marker of FIGS. 4A and 4B in accordance with an embodiment of the present disclosure.

FIG. 4E depicts a perspective view of a third step securing fasteners to the fiducial marker of FIGS. 4A and 4B in accordance with an embodiment of the present disclosure.

FIG. 5A depicts an overhead view of a third embodiment of a fiducial marker configured to be affixed to a bone in accordance with an embodiment of the present disclosure.

FIG. 5B depicts a perspective view of the fiducial marker of FIG. 5A in accordance with an embodiment of the present disclosure.

FIG. 5C depicts a sectional view of the fiducial marker of FIG. 5A along line 5-5 in accordance with an embodiment of the present disclosure.

FIG. 5D depicts a perspective view of a first step securing fasteners to the fiducial marker of FIGS. 5A-C in accordance with an embodiment of the present disclosure.

FIG. 5E depicts a perspective view of a second step securing fasteners to the fiducial marker of FIGS. 5A-C in accordance with an embodiment of the present disclosure.

FIG. 6A depicts a perspective view of a fiducial marker configured to be affixed to a probe in accordance with an embodiment of the present disclosure.

FIG. 6B depicts a reverse perspective view of the fiducial marker of FIG. 6A in accordance with an embodiment of the present disclosure.

FIG. 6C depicts a sectional view of the fiducial marker of FIG. 6A along line 6-6 in accordance with an embodiment of the present disclosure.

FIG. 7A depicts an exploded view of a fiducial marker and a probe in accordance with an embodiment of the present disclosure.

FIG. 7B depicts an elevational view of the fiducial marker and the probe of FIG. 7A secured together in accordance with an embodiment of the present disclosure.

FIG. 8 depicts a perspective view of a probe including a fiducial marker engaged with a cut guide in accordance with an embodiment of the present disclosure.

FIG. 9A depicts a perspective view of a first step of the performance of a femoral distal cut using the probe-containing cut guide in accordance with an embodiment of the present disclosure.

FIG. 9B depicts a perspective view of a second step of the performance of a femoral distal cut using the probe-containing cut guide in accordance with an embodiment of the present disclosure.

FIG. 9C depicts a perspective view of a third step of the performance of a femoral distal cut using the probe-containing cut guide in accordance with an embodiment of the present disclosure.

FIG. 10A depicts a perspective view of a first step of the performance of a tibial proximal cut using the cut guide in accordance with an embodiment of the present disclosure.

FIG. 10B depicts a perspective view of a second step of the performance of a tibial proximal cut using the cut guide in accordance with an embodiment of the present disclosure.

FIG. 10C depicts a perspective view of a third step of the performance of a tibial proximal cut using the cut guide in accordance with an embodiment of the present disclosure.

FIG. 11A depicts a perspective view of a first step of the use of the cut guide for verification of the femoral distal cut in accordance with an embodiment of the present disclosure.

FIG. 11B depicts a perspective view of a second step of the use of the cut guide for verification of the femoral distal cut in accordance with an embodiment of the present disclosure.

FIG. 12A depicts a perspective view of a first step of the use of the cut guide for verification of the tibial proximal cut in accordance with an embodiment of the present disclosure.

FIG. 12B depicts a perspective view of a second step of the use of the cut guide for verification of the tibial proximal cut in accordance with an embodiment of the present disclosure.

FIG. 13A depicts an exploded view of the probe, cut guide, and internal rotation guide in accordance with an embodiment of the present disclosure.

FIG. 13B depicts a perspective view of the probe, cut guide, and internal rotation guide of FIG. 13A secured together in accordance with an embodiment of the present disclosure.

FIG. 14A depicts a perspective view of a first step of the use of the internal rotation guide for placement of the pins for the finishing block on the femoral distal cut in accordance with an embodiment of the present disclosure.

FIG. 14B depicts a perspective view of a second step of the use of the internal rotation guide for placement of the pins for the finishing block on the femoral distal cut in accordance with an embodiment of the present disclosure.

FIG. 14C depicts a perspective view of a third step of the use of the internal rotation guide for placement of the pins for the finishing block on the femoral distal cut in accordance with an embodiment of the present disclosure.

FIG. 15A depicts a perspective view of a plane tool configured to engage with a probe in accordance with an embodiment of the present disclosure.

FIG. 15B depicts a reverse perspective view of the plane tool of FIG. 15A in accordance with an embodiment of the present disclosure.

FIG. 15C depicts a side elevational view of the plane tool of FIG. 15A in accordance with an embodiment of the present disclosure.

FIG. 16A depicts an exploded view of the plane tool of FIG. 15A and a probe in accordance with an embodiment of the present disclosure.

FIG. 16B depicts a perspective view of the plane tool of FIG. 15A and the probe secured together in accordance with an embodiment of the present disclosure.

FIG. 17A depicts a perspective view of the first step of the use of the plane tool for guiding the femoral distal cut in accordance with an embodiment of the present disclosure.

FIG. 17B depicts a perspective view of the second step of the use of the plane tool for guiding the femoral distal cut in accordance with an embodiment of the present disclosure.

FIG. 18A depicts a perspective view of the first step of the use of the plane tool for guiding the tibial proximal cut in accordance with an embodiment of the present disclosure.

FIG. 18B depicts a perspective view of the second step of the use of the plane tool for guiding the tibial proximal cut in accordance with an embodiment of the present disclosure.

FIG. 19A depicts a perspective view of a first step of using the plane tool for verifying the femoral distal cut in accordance with an embodiment of the present disclosure.

FIG. 19B depicts a perspective view of a second step of using the plane tool for verifying the femoral distal cut in accordance with an embodiment of the present disclosure.

FIG. 20A depicts a perspective view of a first step of using the plane tool for verifying the tibial proximal cut in accordance with an embodiment of the present disclosure.

FIG. 20B depicts a perspective view of a second step of using the plane tool for verifying the tibial proximal cut in accordance with an embodiment of the present disclosure.

FIG. 21A depicts an exploded view of the probe-containing plane tool and the internal rotation guide in accordance with an embodiment of the present disclosure.

FIG. 21B depicts a perspective view of the probe-containing plane tool and the internal rotation guide of FIG. 21A secured together in accordance with an embodiment of the present disclosure.

FIG. 22A depicts an exploded view of a probe holder and a probe in accordance with an embodiment of the present disclosure.

FIG. 22B depicts a perspective view of the probe holder and the probe of FIG. 22A secured together in accordance with an embodiment of the present disclosure.

### DETAILED DESCRIPTION

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI is a registered trademark of BLUE BELT TECHNOLOGIES, INC. of Pittsburgh, PA, which is a subsidiary of SMITH & NEPHEW, INC. of Memphis, TN.

### Cass Ecosystem Overview

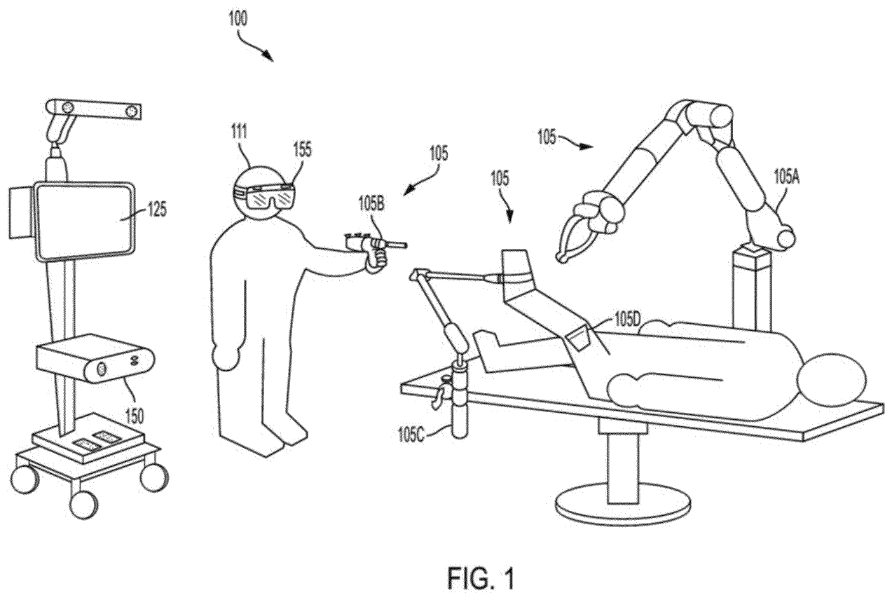

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA) or THA. For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the embodiment employed. For example, for a knee surgery, the Effector Platform 105 may include an End Effector 105B that holds surgical tools or instruments during their use. The End Effector 105B may be a handheld device or instrument used by the surgeon (e.g., a CORI® hand piece or a cutting guide or jig) or, alternatively, the End Effector 105B can include a device or instrument held or positioned by a robotic arm 105A. While one robotic arm 105A is illustrated in FIG. 1, in some embodiments there may be multiple devices. As examples, there may be one robotic arm 105A on each side of an operating table T or two devices on one side of the table T. The robotic arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or alternatively change limb positions based on instructions received from the Surgical Computer 150 (described below). While one Limb Positioner 105C is illustrated in FIG. 1, in some embodiments there may be multiple devices. As examples, there may be one Limb Positioner 105C on each side of the operating table T or two devices on one side of the table T. The Limb Positioner 105C may be mounted directly to the table T, be located next to the table T on a floor platform (not shown), mounted on a pole, or mounted on a wall or ceiling of an operating room. In some embodiments, the Limb Positioner 105C can be used in non-conventional ways, such as a retractor or specific bone holder. The Limb Positioner 105C may include, as examples, an ankle boot, a soft tissue clamp, a bone clamp, or a soft-tissue retractor spoon, such as a hooked, curved, or angled blade. In some embodiments, the Limb Positioner 105C may include a suture holder to assist in closing wounds.

The Effector Platform 105 may include tools, such as a screwdriver, light or laser, to indicate an axis or plane, bubble level, pin driver, pin puller, plane checker, pointer, finger, or some combination thereof.

Resection Equipment 110 (not shown in FIG. 1) performs bone or tissue resection using, for example, mechanical, ultrasonic, or laser techniques. Examples of Resection Equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the Resection Equipment 110 is held and operated by the surgeon during surgery. In other embodiments, the Effector Platform 105 may be used to hold the Resection Equipment 110 during use.

The Effector Platform 105 also can include a cutting guide or jig 105D that is used to guide saws or drills used to resect tissue during surgery. Such cutting guides 105D can be formed integrally as part of the Effector Platform 105 or robotic arm 105A or cutting guides can be separate structures that can be matingly and/or removably attached to the Effector Platform 105 or robotic arm 105A. The Effector Platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 also can be used to infer velocity/acceleration of anatomy/instrumentation, which can be used for tool control. In some embodiments, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various embodiments of the present invention including. without limitation, Infrared (IR) tracking systems, electromagnetic (EM) tracking systems, video or image based tracking systems, and ultrasound registration and tracking systems. Using the data provided by the tracking system 115, the surgical computer 150 can detect objects and prevent collision. For example, the surgical computer 150 can prevent the robotic arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the robotic arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one-or two-dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A Tissue Navigation System 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The Display 125 provides graphical user interfaces (GUIs) that display images collected by the Tissue Navigation System 120 as well other information relevant to the surgery. For example, in one embodiment, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical Computer 150 provides control instructions to various components of the CASS 100, collects data from those components, and provides general processing for various data needed during surgery. In some embodiments, the Surgical Computer 150 is a general-purpose computer. In other embodiments, the Surgical Computer 150 may be a parallel computing platform that uses multiple central processing units (CPUs) or graphics processing units (GPU) to perform processing. In some embodiments, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

### Robotic Arm

In some embodiments, the CASS 100 includes a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some embodiments, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In embodiments in which an end effector 105B is used, the end effector may be positioned at the end of the robotic arm 105A such that any motor control operations are performed within the robotic arm system. In embodiments in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some embodiments, the robotic arm 105A can be moved by hand (e.g., by the surgcon) or with internal motors into its ideal position and orientation for the task being performed. In some embodiments, the robotic arm 105A may be enabled to operate in a “free” mode that allows the surgeon to position the arm into a desired position without being restricted. While in the free mode, the position and orientation of the robotic arm 105A may still be tracked as described above. In one embodiment, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 also can place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

A robotic arm 105A may be used for holding the retractor. For example, in one embodiment, the robotic arm 105A may be moved into the desired position by the surgeon. At that point, the robotic arm 105A may lock into place. In some embodiments, the robotic arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The robotic arm 105A may also be used to help stabilize the surgeon's hand while making a femoral neck cut. In this application, control of the robotic arm 105A may impose certain restrictions to prevent soft tissue damage from occurring. For example, in one embodiment, the Surgical Computer 150 tracks the position of the robotic arm 105A as it operates. If the tracked location approaches an area where tissue damage is predicted, a command may be sent to the robotic arm 105A causing it to stop. Alternatively, where the robotic arm 105A is automatically controlled by the Surgical Computer 150, the Surgical Computer may ensure that the robotic arm is not provided with any instructions that cause it to enter areas where soft tissue damage is likely to occur. The Surgical Computer 150 may impose certain restrictions on the surgeon to prevent the surgeon from reaming too far into the medial wall of the acetabulum or reaming at an incorrect angle or orientation.

In some embodiments, the robotic arm 105A may be used to hold a cup impactor at a desired angle or orientation during cup impaction. When the final position has been achieved, the robotic arm 105A may prevent any further seating to prevent damage to the pelvis.

The surgeon may use the robotic arm 105A to position the broach handle at the desired position and allow the surgeon to impact the broach into the femoral canal at the desired orientation. In some embodiments, once the Surgical Computer 150 receives feedback that the broach is fully seated, the robotic arm 105A may restrict the handle to prevent further advancement of the broach.

The robotic arm 105A may also be used for resurfacing applications. For example, the robotic arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. patent application Ser. No. 15/525,585 to Krinninger et al., U.S. patent application Ser. No. 15/561,042 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention, the episode of care can include three phases: pre-operative, intra-operative, and post-operative. During cach phase, data is collected or generated that can be used to analyze the episode of care in order to understand various features of the procedure and identify patterns that may be used, for example, in training models to make decisions with minimal human intervention. The data collected over the episode of care may be stored at the Surgical Computer 150 or the Surgical Data Server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the Surgical Computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

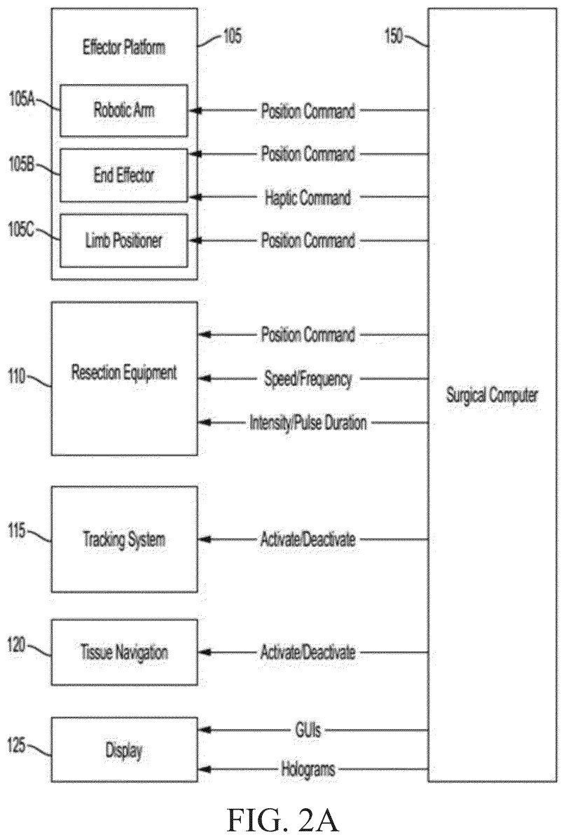

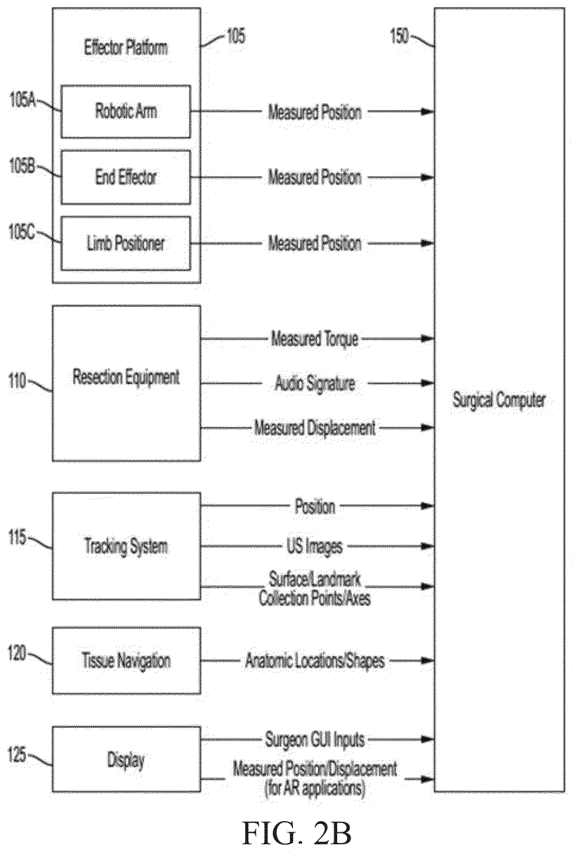

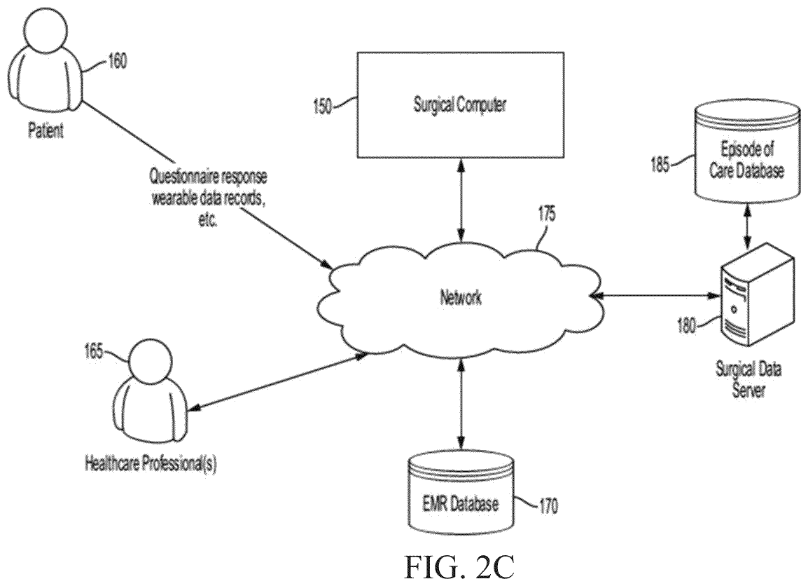

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the Surgical Computer 150 provides to other components of the CASS 100, according to some embodiments. Note that the example of FIG. 2A assumes that the components of the Effector Platform 105 are each controlled directly by the Surgical Computer 150. In embodiments where a component is manually controlled by the Surgeon 111, instructions may be provided on the Display 125 or AR HMD 155 instructing the Surgeon 111 how to move the component.

The various components included in the Effector Platform 105 are controlled by the Surgical Computer 150 providing position commands that instruct the component where to move within a coordinate system. In some embodiments, the Surgical Computer 150 provides the Effector Platform 105 with instructions defining how to react when a component of the Effector Platform 105 deviates from a surgical plan. These commands are referenced in FIG. 2A as “haptic” commands. For example, the End Effector 105B may provide a force to resist movement outside of an area where resection is planned. Other commands that may be used by the Effector Platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The Resection Equipment 110 is provided with a variety of commands to perform bone or tissue operations. As with the Effector Platform 105, position information may be provided to the Resection Equipment 110 to specify where it should be located when performing resection. Other commands provided to the Resection Equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the Surgical Computer 150; rather, the Surgical Computer 150 only needs to activate the component, which then executes software locally specifying the manner in which to collect data and provide it to the Surgical Computer 150. In the example of FIG. 2A, there are two components that are operated in this manner: the Tracking System 115 and the Tissue Navigation System 120.

The Surgical Computer 150 provides the Display 125 with any visualization that is needed by the Surgeon 111 during surgery. For monitors, the Surgical Computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information conceming rotation and alignment of the joint. The display 125 can depict how the planned implants' locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon 111 who typically performs the tibial cuts before the femoral cuts in a TKA, the display 125 and associated workflow can be adapted to take this preference into account. The surgeon I11 also can preselect that certain steps be included or deleted from the standard surgical workflow display. For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre-and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the Surgical Computer 150 may provide images, text, etc. using the data format supported by the equipment. For example, if the Display 125 is a holography device such as the Microsoft HoloLensTM or Magic Leap One™, the Surgical Computer 150 may use the HoloLens Application Program Interface (API) to send commands specifying the position and content of holograms displayed in the field of view of the Surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD™ simulation software from SMITH AND NEPHEW, INC. In some embodiments, the Surgical Computer 150 includes computing architecture that allows full execution of the surgical planning model during surgery (e.g., a GPU-based parallel processing environment). In other embodiments, the Surgical Computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the Surgical Computer 150 from the various components of the CASS 100. In some embodiments, the components may stream data to the Surgical Computer 150 in real-time or near real-time during surgery. In other embodiments, the components may queue data and send it to the Surgical Computer 150 at set intervals (e.g., every second). Data may be communicated using any format known in the art. Thus, in some embodiments, the components all transmit data to the Surgical Computer 150 in a common format. In other embodiments, each component may use a different data format, and the Surgical Computer 150 is configured with one or more software applications that enable translation of the data.

In general, the Surgical Computer 150 may serve as the central point where CASS data is collected. The exact content of the data will vary depending on the source. For example, cach component of the Effector Platform 105 provides a measured position to the Surgical Computer 150. Thus, by comparing the measured position to a position originally specified by the Surgical Computer 150 (see FIG. 2B), the Surgical Computer can identify deviations that take place during surgery.

The Resection Equipment 110 can send various types of data to the Surgical Computer 150 depending on the type of equipment used. Example data types that may be sent include the measured torque, audio signatures, and measured displacement values. Similarly, the Tracking Technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the Surgical Computer 150 with anatomic locations, shapes, etc. as the system operates.

Although the Display 125 generally is used for outputting data for presentation to the user, it may also provide data to the Surgical Computer 150. For example, for embodiments where a monitor is used as part of the Display 125, the Surgeon 111 may interact with a GUI to provide inputs which are sent to the Surgical Computer 150 for further processing. For AR applications, the measured position and displacement of the HMD may be sent to the Surgical Computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the Surgical Computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the Surgical Computer 150 or other CASS component by the patient or the patient's physician(s). Alternatively, or additionally, the Surgical Computer 150 or other CASS component can monitor the patient's EMR and retrieve relevant information as it becomes available. This longitudinal view of the patient's recovery allows the Surgical Computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the Surgical Computer 150 into order to retrieve data collected during surgery. The data can then be transferred, for example, via a desktop computer to the centralized storage. Alternatively, in some embodiments, the Surgical Computer 150 is connected directly to the centralized storage via a Network 175 as shown in FIG. 2C.

FIG. 2C illustrates a “cloud-based” implementation in which the Surgical Computer 150 is connected to a Surgical Data Server 180 via a Network 175. This Network 175 may be, for example, a private intranet or the Internet. In addition to the data from the Surgical Computer 150, other sources can transfer relevant data to the Surgical Data Server 180. The example of FIG. 2C shows three additional data sources: the Patient 160, Healthcare Professional(s) 165, and an EMR Database 170. Thus, the Patient 160 can send pre-operative and post-operative data to the Surgical Data Server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the Surgical Computer 150 may use data from past surgeries, machine learning models, etc. to help guide the surgical procedure.

In some embodiments, the Surgical Computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the Surgical Computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the Surgical Computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data are described in U.S. Patent Application No. Ser. No. 16/847,183, filed Apr. 13, 2020, published as U.S. Publication No. 2020/0243199, and entitled “METHODS AND SYSTEMS FOR PROVIDING AN EPISODE OF CARE,” the entirety of which is incorporated herein by reference.

### Fiducial Marker Assemblies for Navigated Surgical Procedures

Described herein are fiducial marker assemblies for use in conjunction with a surgical navigation system or CASS, such as are described above, for the performance of a navigated surgical procedure, such as a TKA. One issue with conventional fiducial markers is that they can have blind spots in particular orientations, i.e., there can be orientations relative to the navigation system where the fiducials may not be identifiable thereby. This can be an issue in surgical procedures because it can be time-consuming to properly place fiducial markers and ensure that they are all individually identifiable by the navigation system.

Further, the operating room is a dynamic environment, and a fiducial marker that is initially properly oriented with respect to the navigation system may become moved or mis-oriented during the course of the procedure. In contrast, the fiducial markers described herein are designed to be identifiable by a surgical navigation system from substantially any angle. Therefore, these fiducial markers are more efficient to set up and more robustly account for movements during the surgical procedure, which reduces the potential for intraoperative navigation errors and minimizes the need to periodically adjust and re-register the fiducial markers with the navigation system.

The fiducial marker assemblies described herein could be used for image-free orthopedic navigated surgery (e.g., a TKA). The fiducial markers are designed to assist the surgeon in controlling surgical variables for obtaining optimal implant alignment by increasing the operational efficiency and preventing navigation errors due to mis-identified fiducials. The fiducial marker assemblies could also be used in image-based orthopedic procedures, wherein a patient-specific 3D bone model is used to match the patient's anatomy, as opposed to using bony landmarks.

In some embodiments, some or all of the instrumentation described herein can be disposable, which could represent a commercial and/or clinical advantage over conventional systems that rely on equipment that is expensive to clean and/or store. In some embodiments, the instrumentation described herein could be implant-agnostic, which could represent a commercial and/or clinical advantage over conventional systems that have limited or no interoperability (i.e., are manufacturer-specific). The surgical equipment kit described herein can include fiducial markers that can be attached to the femur and tibia of the patient using conventional fasteners (e.g., 3.2 mm Steinmann pins). The probe (which could be pre-calibrated from the factory) could be used to sample anatomical landmarks. The probe could thereafter be inserted into the cut guide (which, once again, could be pre-calibrated from the factory) to provide guidance to the surgeon on where to perform the desired cuts. Finally, the internal rotation guide could be secured to the cut guide and the probe to guide the drilling of the holes for the femoral finishing block. The surgical equipment kit described herein can utilize the same fiducial markers to perform a complete surgical procedure (e.g., a TKA) without the need to individually register and calibrate each component of the surgical equipment kit with the surgical navigation system.

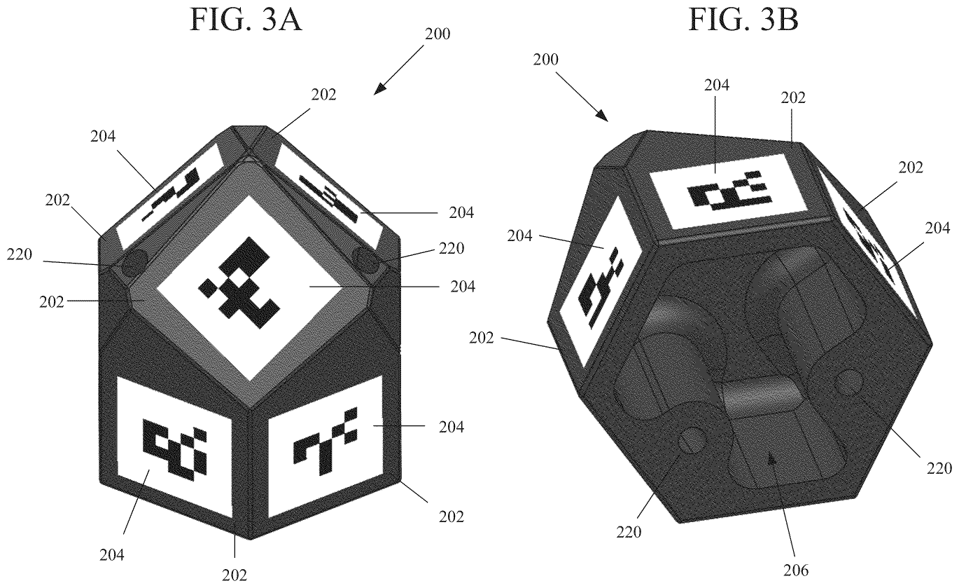

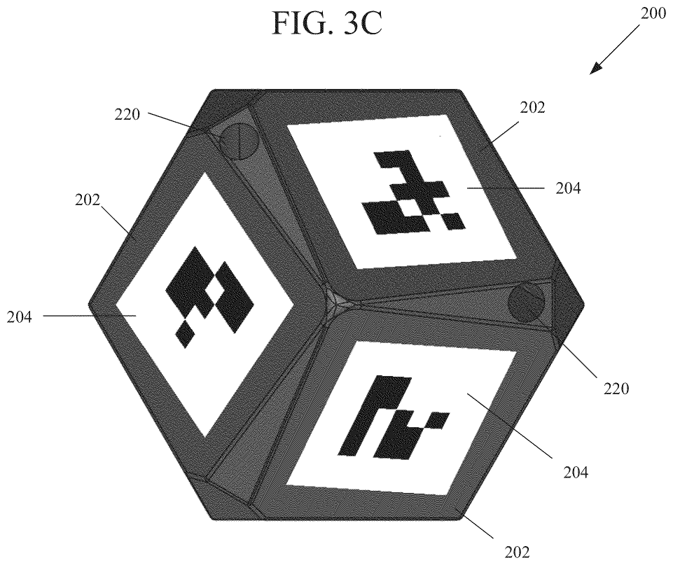

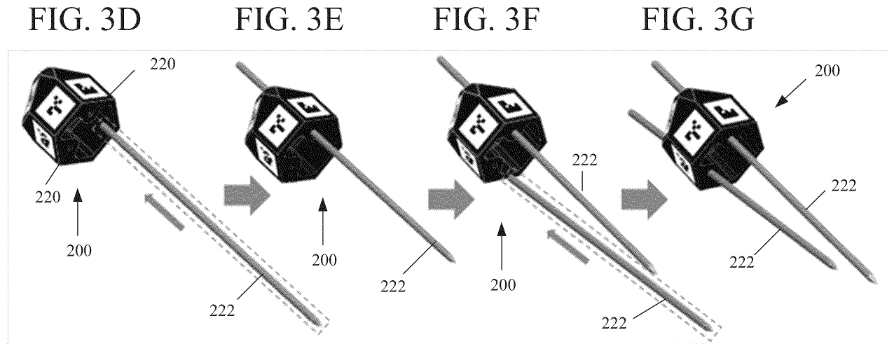

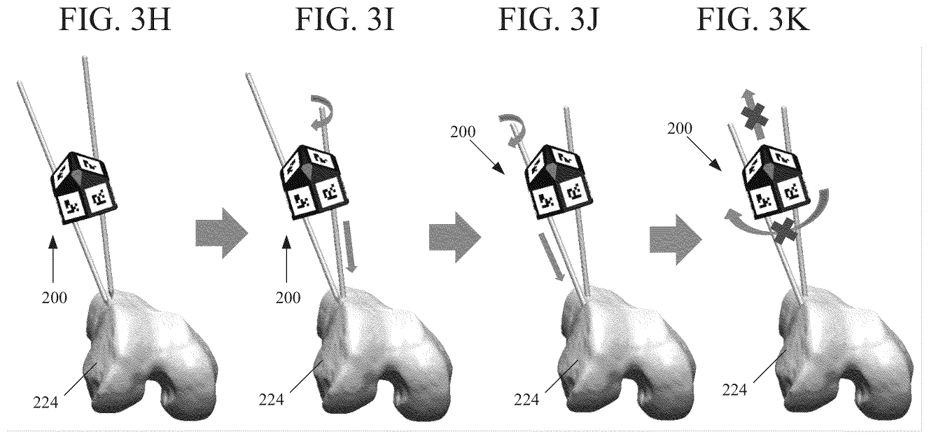

The fiducial markers described herein can be configured to be viewable from substantially any orientation. Stated differently, the fiducial markers can lack any “blind spots” that prevent less than two fiducials from being visible from a particular orientation. In various embodiments shown in FIGS. 3A-6C, the fiducial markers 200 include multiple faces 202 and each of the faces 202 include a fiducial 204. In one embodiment shown in FIGS. 3A-C, the fiducial marker 200 can include a combination of a hexagonal prism body portion and a triangular prism top portion. In this embodiment, the fiducial marker 200 includes nine faces 202 (six faces for the hexagonal prism body portion and three faces from the triangular prism top portion) that each bear a fiducial 204. The illustrated embodiments for the fiducial marker 200 are provided simply for illustrative purposes and other embodiments could be arranged in a variety of other shapes and configurations. In all of these embodiments, the fiducial marker 200 is configured such that at least two of the faces 202 (and, thus, the corresponding fiducials 204) are visible from substantially any orientation, except from a straight-on bottom view (where none of the faces 202 would be visible). Because at least two faces 202 of the fiducial markers 200 are visible from substantially any orientation, the various embodiments of the fiducial marker 200 are more efficient to utilize intraoperatively because they do not need to be placed and oriented in precise manner to ensure that they are properly registered by the tracking system 115 and, further, more robustly account for movement within the operating room (e.g., movement of the patient by the surgical team or movement of the tracking system 115 during the procedure). In particular, the various configurations of the fiducial markers 200 maximize the visibility of more than one face 202, minimize the occurrences of particular orientations that increase the chance for errors due to homography estimation of the planar faces, and minimize of the presence of over-slanted faces 202 in particular orientations.

In any of the embodiments, the fiducials 204 can include, for example, passive fiducials that are identifiable by the tracking system 115 (FIGS. 2A and 2B) of a surgical navigation system. Passive fiducials could include reflective fiducials (e.g., infrared-reflective fiducials), QR-code based fiducials, and any other type of fiducials that are identifiable by a surgical navigation system. In other embodiments, the fiducials 204 could include active fiducials. In some embodiments, the fiducial markers 200 can be constructed from a biocompatible material (e.g., a biocompatible plastic).

In some embodiments, the fiducials 204 can further be utilized in an augmented reality (AR) system. In particular, the fiducials 204 could be individually identifiable, allowing for information to be rendered over the anatomy in real-time via an AR-enabled display device (e.g., a head-mounted display, a smartphone, or a tablet). In other embodiments, the fiducials 204 could be utilized in an endoscopic surgical system. In particular, individually identifiable fiducials 204 could be identified by the surgical system and real-time information could be rendered on the display of the endoscope over the anatomy being visualized thereby.

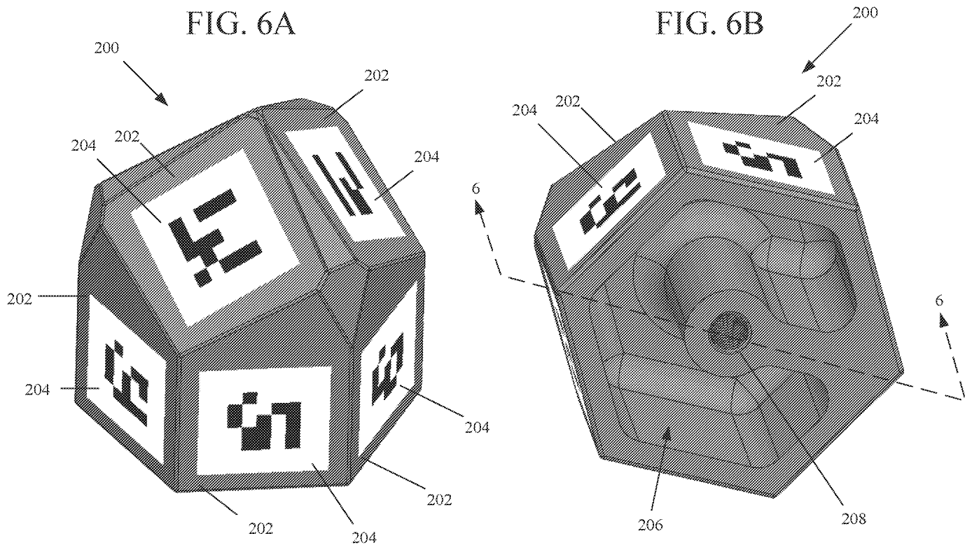

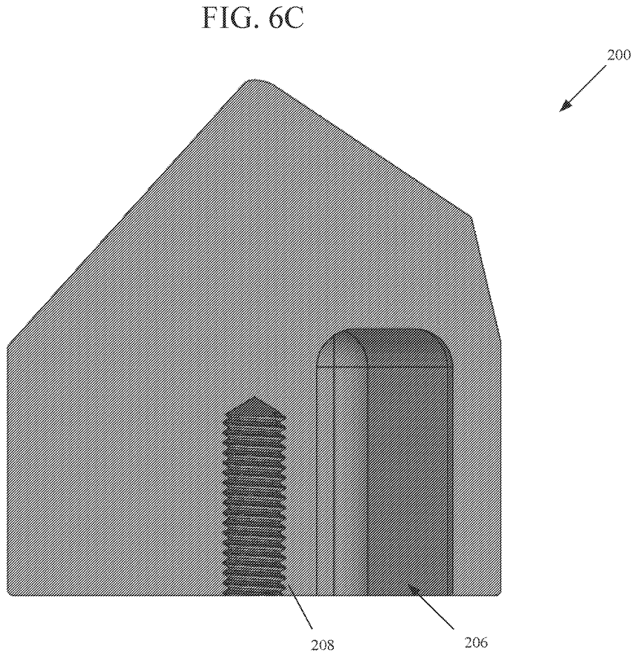

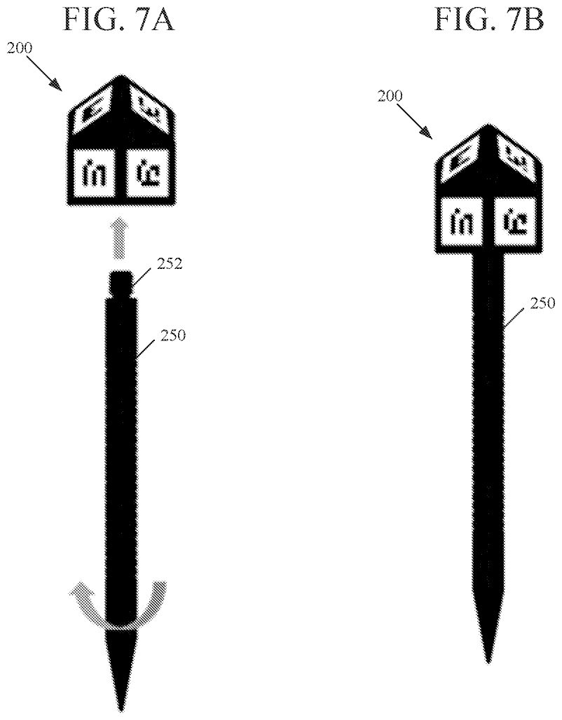

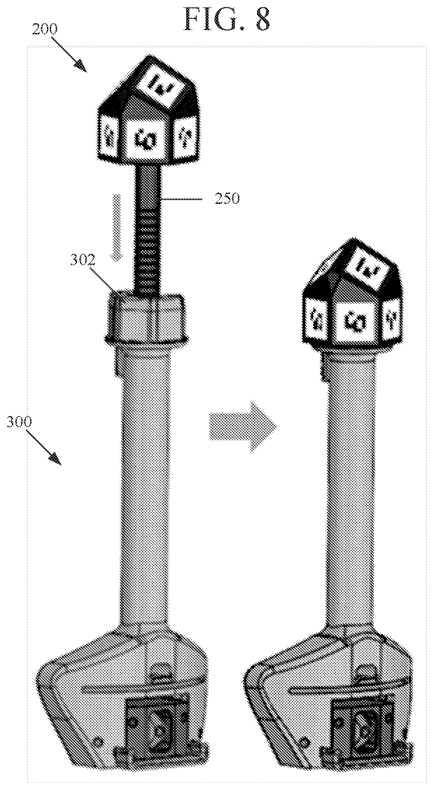

The fiducial markers 200 can include a variety of different configurations that allow them to be attached to different devices and utilized for different purposes. For example, FIGS. 3A-5E illustrate embodiments of fiducial markers 200 that are designed to be affixed to patients' bones (e.g., the tibia and/or femur) and FIGS. 6A-7B illustrate an embodiment of a fiducial marker 200 that is designed to be affixed to a probe 250 (which can in turn be used in combination with a number of other devices, as shown in FIGS. 8-22B and described below). The various embodiments of bone and probe-affixed fiducial markers 200 are described below.

Conventional fiducial markers are generally intraoperatively affixed to a patient's bone via a single fastener. However, this single-pin attachment technique creates issues because the fiducial marker could be moved or reoriented if torsional forces are applied to the fiducial marker, which in turn can cause the fiducial marker to no longer be properly oriented with respect to the tracking system 115 and can force the surgical team to re-register the fiducial marker(s) with the surgical navigation system. This process, of course, is inefficient and delays the surgical procedure. Therefore, the embodiments of the fiducial markers 200 that are designed to be affixed to a patient's bone 224 are configured such that they will not rotate or otherwise reorient intraoperatively in response to torsional forces applied thereto.

In one embodiment shown in FIGS. 3A-3K, a fiducial marker 200 configured to be affixed to a bone 224 via fasteners 222 can include two or more channels 220 that are arranged at angles with respect to each other. The channels 220 can be configured to receive a fastener 222 therethrough, such as a Steinman pin. The channels 220 extend through the body of the fiducial marker 200 and are angled with respect to each so that the fasteners 222 inserted therethrough are oriented at corresponding angles with respect to each other. As shown in FIGS. 3H-3K, the fiducial marker 200 can be affixed to the patient's bone 224 via fasteners 222 inserted through each of the channels 220. Because the channels 220 are oriented at an angle with respect to each other, the fiducial marker 200 is affixed in place in a manner that prevents both translational and rotational movement of the fiducial marker 200 relative to the bone 224, as shown in FIG. 3K. In some implementations, the fasteners 222 could be attached backwards into the fiducial marker 200 to avoid causing debris from the sharp tip of the fastener 222 drilling into the fiducial marker 200. Due to the stiffness of the fasteners 222, friction, and the fact that the orientations of the fasteners 222 are not on the same plane, the fasteners 222 stabilize the fiducial marker 200 and accordingly secure the fiducial marker 200 in position relative to the bone 224.

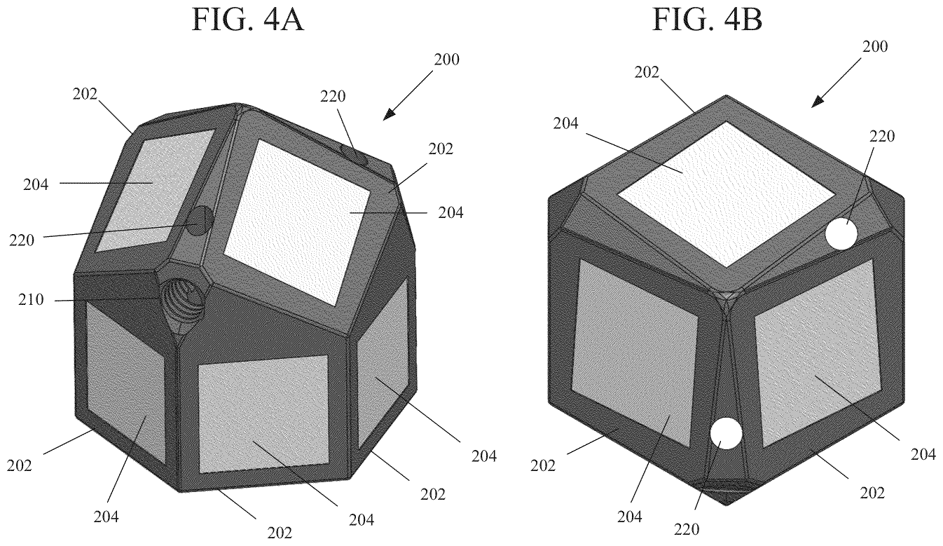

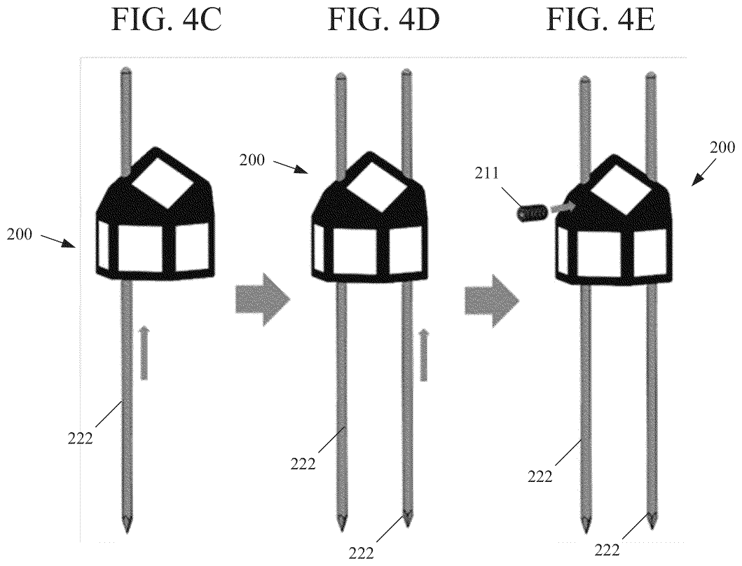

In another embodiment shown in FIGS. 4A-4E, a fiducial marker 200 configured to be affixed to a bone 224 via fasteners 222 can include a threaded aperture 210 that is configured to receive a set screw 211 therethrough. The threaded aperture 210 can be oriented generally transversely with respect to the channels 220. Further, the threaded aperture 210 intersects with one or more of the fastener channels 220 such that the set screw 211 inserted through the threaded aperture 210 can frictionally engage with a fastener 222 inserted therethrough, as shown in FIG. 4E. When frictionally engaged with the fastener 222. the set screw 211 can fix the fiducial marker 200 at the desired height and prevent the fiducial marker 200 from rotating with respect to the fasteners 222. Accordingly, the set screw 211 can be used as an alternative or additional mechanism for securing the fiducial marker 200 in place along the fasteners 222 and, thereby, relative to the bone 224. In this embodiment, the channels 220 can be angled, as in the embodiment shown in FIGS. 3A-3K, or oriented parallel with respect to each other, as shown in FIGS. 4C-4E. This embodiment configured to be used in conjunction with a set screw 211 can be beneficial because the use of the set screw 211 could further increase the stability of the fiducial marker 200 to movements caused by impacts or vibrations from other tools.

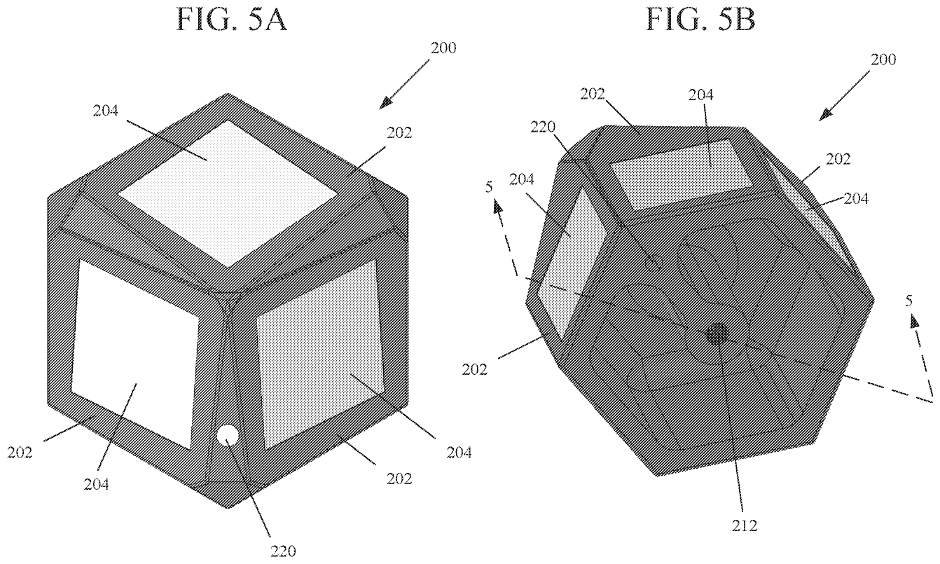

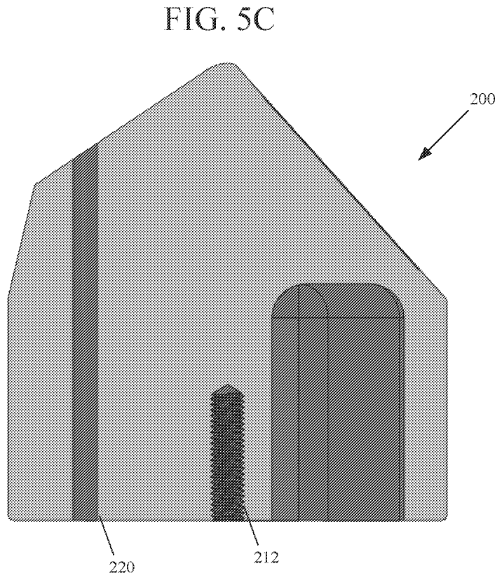

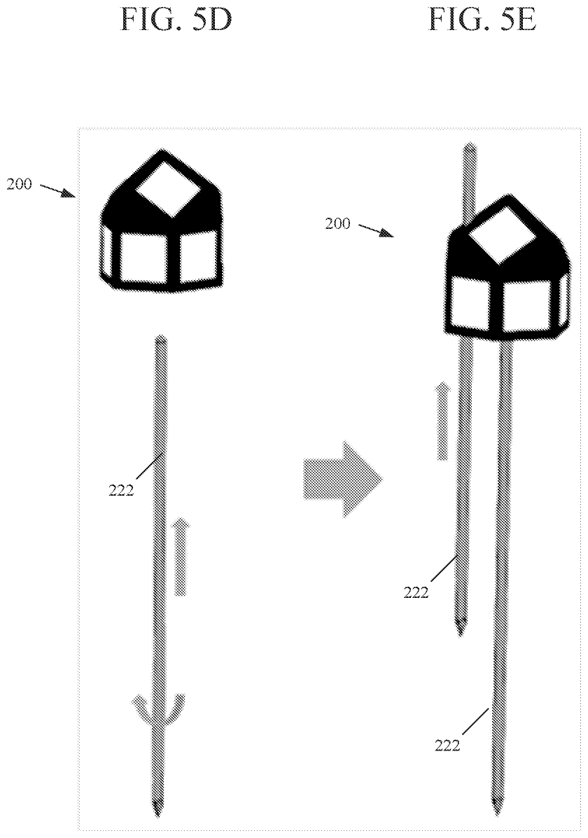

In another embodiment shown in FIGS. 5A-5E, a fiducial marker 200 configured to be affixed to a bone 224 via fasteners 222 can include a threaded channel 212 that is configured to engage with a corresponding threaded fastener 223. In this embodiment, the threaded channel 212 can replace one or more of the channels 220 described above with respect to the embodiments shown in FIGS. 3A-4E. The threaded channel 212 can be configured to engage with a corresponding threaded portion of a fastener 222. By threadably engaging at least one of the fasteners 222 with the fiducial marker 200, the fiducial marker 200 can be fixed at the desired height and prevented from rotating with respect to the fasteners 222, as shown in FIG. 5E.

As noted above, different embodiments of the fiducial markers 200 can be intended for different intraoperative purposes. The embodiments of the fiducial markers 200 described above with respect to FIGS. 3A-5E can be intended to be intraoperatively affixed to a patient's bone 224. However, alternative embodiments of the fiducial markers 200 can be intended to be intraoperatively affixed to other devices. For example, FIGS. 6A-6C illustrate an embodiment of a fiducial marker that is designed to be affixed to a probe 250, as shown in FIGS. 7A and 7B. In this embodiment, the fiducial markers 200 can further include a recessed interior 206 and a connector 208 that is configured to engage with the probe 250. The probe 250 could include, for example, a point probe as described above. In one embodiment, the connector 208 could include threading that is configured to engage with a corresponding threaded portion 252 on the probe 250. In other embodiments, the connector 208 could include a friction fit connector, snap fit connectors, and so on.

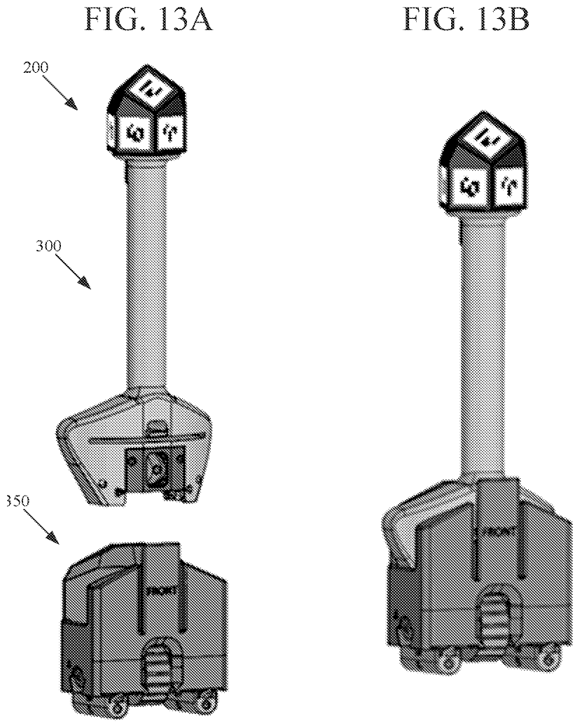

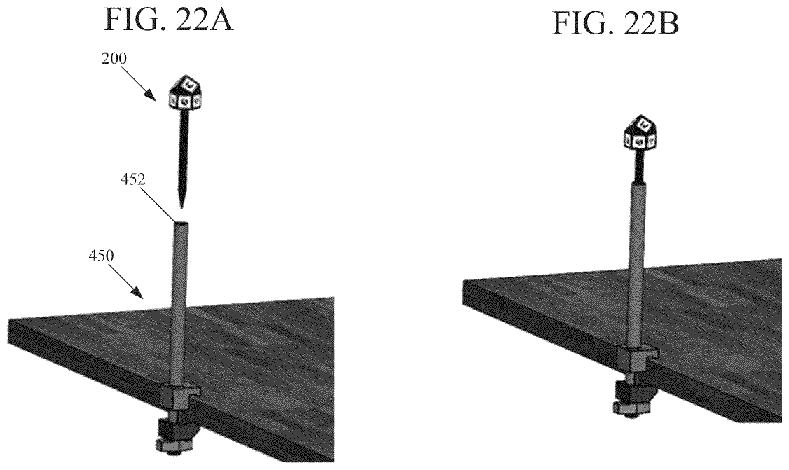

The probe 250 can further be configured to engage with a variety of other components of the surgical system, thereby allowing the probe 250 with the fiducial marker 200 affixed thereto to be used throughout the surgical procedure without the need to separately register each individual piece of surgical equipment. Therefore, the surgical team would only need to register a single piece of equipment, i.e., the probe 250, and could reuse the registered probe 250 for all of the other pieces of surgical equipment. Not having to separately register each individual piece of surgical equipment greatly increases the efficiency of performing the surgical procedure. Further, the ability to reuse a single fiducial marker 200 by alternatively attaching the probe 250 to different pieces of equipment (as described in greater detail below) obviates the need for separate fiducial markers 200 to be affixed to each piece of surgical equipment, which greatly decreases the overall cost of the surgical equipment kit because the fiducials 204 are generally one of, if not the most, expensive components of the surgical equipment kit. The probe 250 could be configured to engage with, for example, a cut guide 300 (FIGS. 8-12B), an internal rotation guide 350 (FIGS. 13A-1C), a plane tool 400 (15A-21B), and/or a probe holder 450 (FIGS. 22A and 22B).

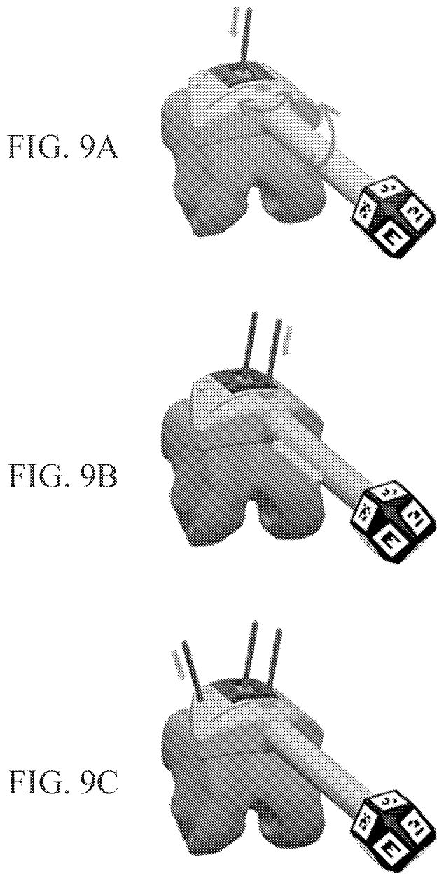

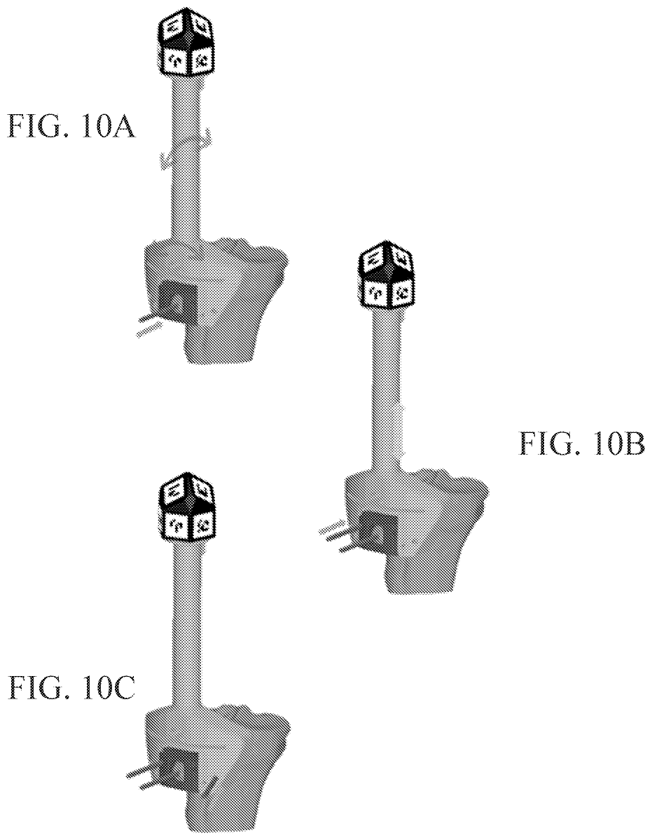

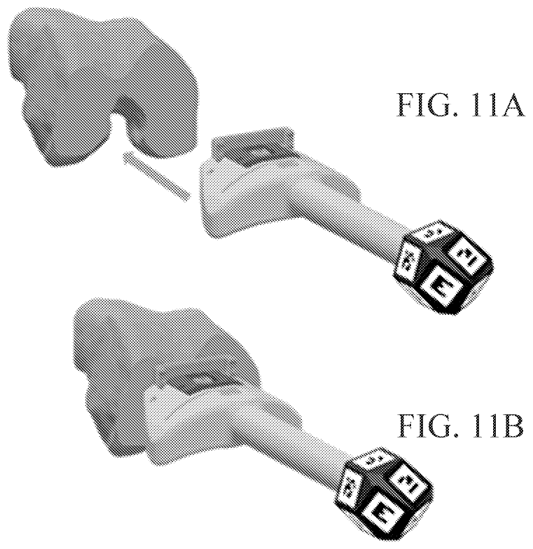

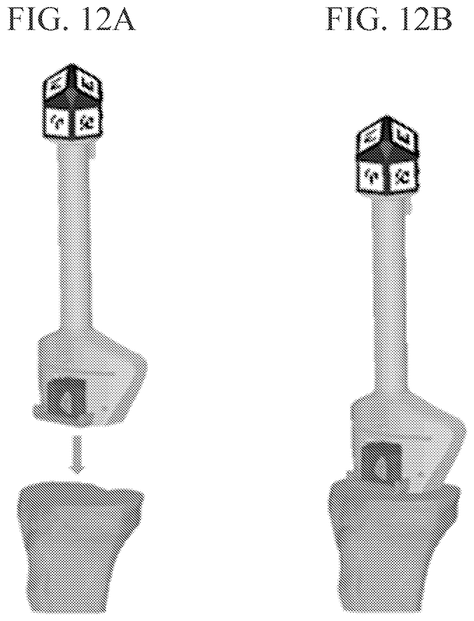

For example, FIGS. 8-12B illustrate a cut guide 300 including a slot 302 or recess that is configured to receive the length of body portion of the probe 250 therein. In some embodiments, the slot 302 can include threading that is configured to engage with corresponding threading on the probe 250. Other embodiments can utilize other mechanisms for securing the probe 250 to the cut guide 300. The cut guide 300 can be utilized for both femoral and tibial bone resections, as well as being used to guide the positioning of the finishing block for the anterior, posterior, and oblique femoral resections. Furthermore, it can be used to record the performed cuts, allowing the operator to rectify an execution error or to tweak the following cuts. If the probe 250 has already been intraoperatively registered with the surgical navigation system, the cut guide 300 does not need to be re-registered with the surgical navigation system. Once the probe 250 is secured to the cut guide 300, the cut guide 300 can thereafter be utilized to perform a number of different surgical cuts or other surgical steps. For example, FIGS. 9A-9C illustrate the performance of a femoral distal cut using the probe-containing cut guide 300, FIGS. 10A-10C illustrate the performance of a tibial proximal cut using the cut guide 300, FIGS. 11A and 11B illustrate the use of the cut guide 300 for verification of the femoral distal cut, and FIGS. 12A and 12B illustrate the use of the cut guide 300 for verification of the tibial proximal cut.

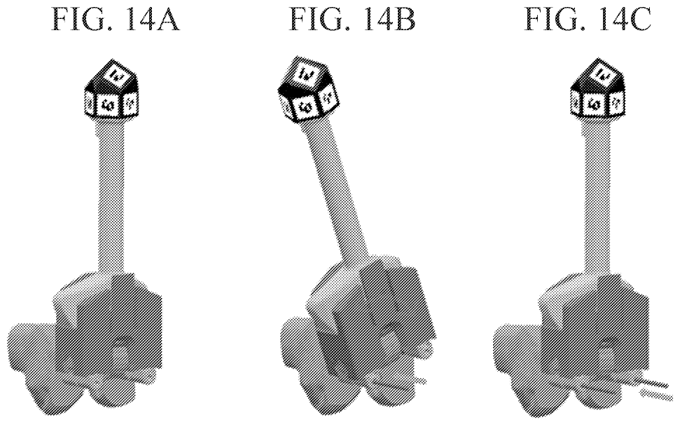

As another example, FIGS. 13A-14C illustrate an internal rotation guide 350 that is configured to engage with the probe 250. In one embodiment, the internal rotation guide 350 could include a 352 or recess that is configured to receive the length of body portion of the probe 250 therein. In the illustrated embodiment, the internal rotation guide 350 is configured to engage with the body portion of the cut guide 300. To perform the anterior, posterior, and oblique cuts, a finishing block is used. The placement of the finishing block is crucial to achieve the desired ligament gap and internal rotation. The internal rotation guide 350 is utilized to guide the placement of the finishing block pins in the distal cut. Using the internal rotation guide 350 is critical to guide the placement of the finishing block pins because the pins are spaced at a distance that is not constant across manufacturers; therefore, the internal rotation guide 350 must precisely guide the placement of the finishing block pins. Once the probe 250 is secured to the internal rotation guide 350, the internal rotation guide 350 can thereafter be utilized to perform a number of different surgical cuts or other surgical steps. For example, FIGS. 14A-14C illustrate the use of the internal rotation guide 350 for placement of the pins for the finishing block on the femoral distal cut.

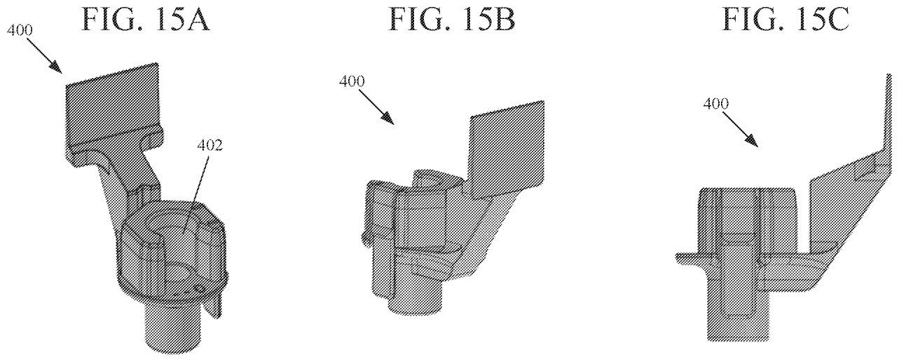

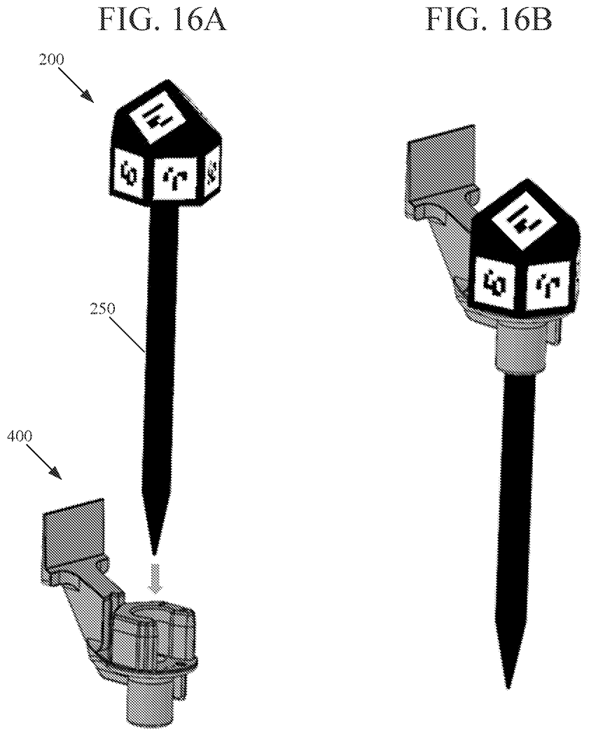

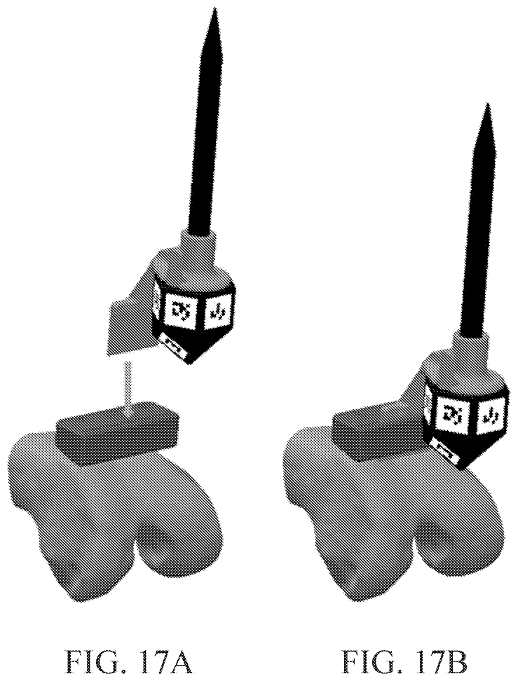

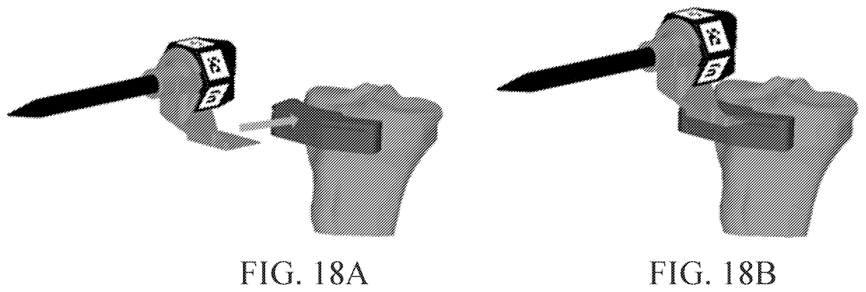

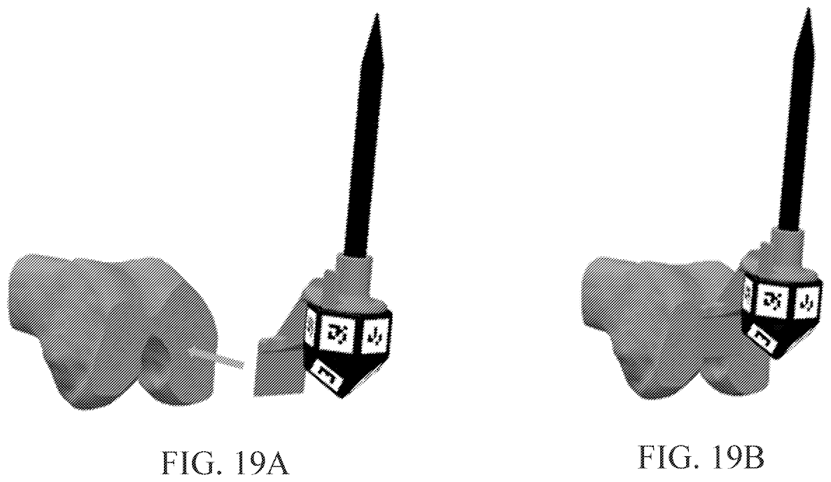

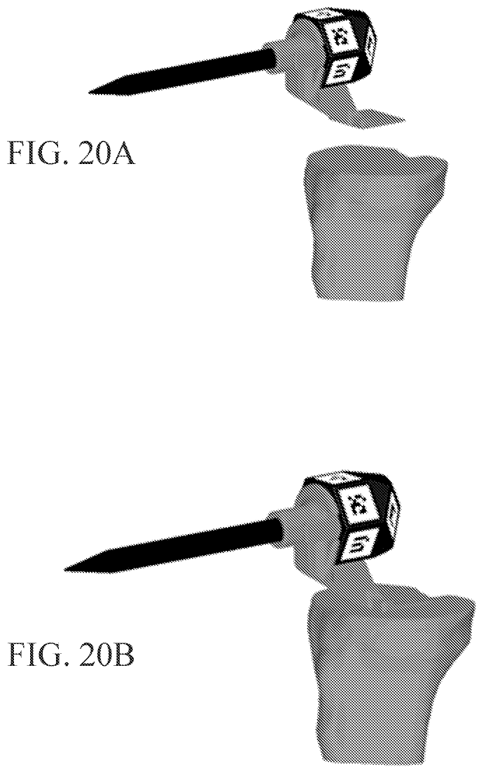

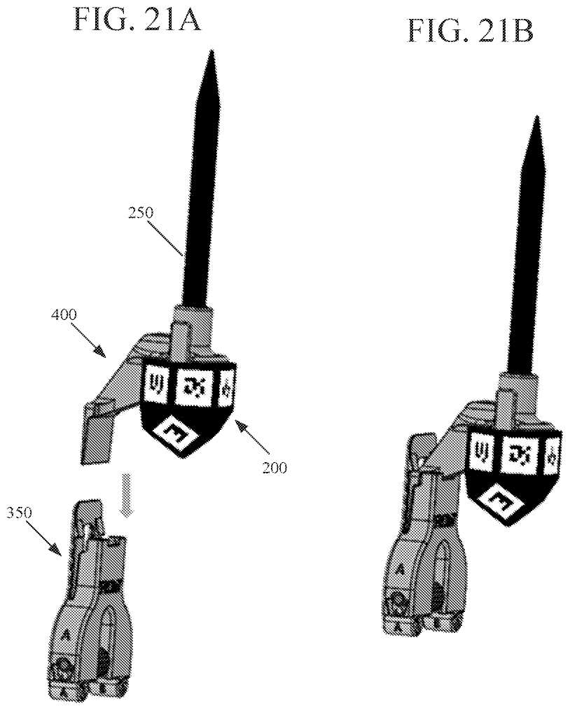

As another example, FIGS. 15A-21B illustrate a plane tool 400 that is configured to engage with the probe 250. The plane tool 400 can include a slot 402 or recess that is configured to receive the length of body portion of the probe 250 therein, as shown in FIGS. 16A and 16B. In some embodiments, the slot 402 can include threading that is configured to engage with corresponding threading on the probe 250. Other embodiments can utilize other mechanisms for securing the probe 250 to the plane tool 400. The plane tool 400, similarly to the cut guide 300, is designed to guide the distal and/or proximal cuts, perform bone resection verifications, and guide the placement of the finishing block pins in the distal cut. FIGS. 17A-18B illustrate the use of the plane tool 400 with the probe 250 secured thereto for guiding the femoral distal cut (FIGS. 17A and 17B) and the tibial proximal cut (FIGS. 18A and 18B). For the distal and proximal cuts, the plane tool 400 is used in conjunction with standard cutting guides. After inserting the plane tool 400 into the cutting guide slots, the operator can adjust the positioning of the cutting guide. When the desired positioning is reached, the operator can secure the cutting guide and the plane tool 400 in place by hand and drill two pins to secure the cutting guide against the bone surface. With the fiducial marker 200 and the probe 250 affixed thereto, these steps can accordingly be done under proper navigation by the surgical system. FIGS. 19A-20B illustrate the user of the plane tool 400 with the probe 250 secured thereto for verifying the femoral distal cut (FIGS. 19A and 19B) and the tibial proximal cut (FIGS. 20A and 20B). For the bone resection verification steps, the planar face of the plane tool 400 can be positioned against the resected surfaces of the bones (i.e., the tibia and femur). Because the planar face of the plane tool 400 is pre-calibrated prior to use, any deviation between the actual cut made intraoperatively and the planned cut can be measured. Finally, the plane tool 400 can also be used in conjunction with an internal rotation guide 350, as shown in FIGS. 21A and 21B. In this embodiment, the internal rotation guide 350 functions generally as described above, except that the internal rotation guide 350 is configured to receive the plane tool 400 therein (as opposed to the body portion of the cut guide 300, as described in the aforementioned embodiment of the internal rotation guide 350).

As yet another example, FIGS. 22A and 22B illustrate a probe holder 450 that is configured to receive the probe 250. The probe holder 450 can include a slot 452 or recess that is configured to receive the length of body portion of the probe 250 therein. In some embodiments, the slot 452 can include threading that is configured to engage with corresponding threading on the probe 250. Other embodiments can utilize other mechanisms for securing the probe 250 to the plane tool 400. The probe holder 450 could include a clamp or other mechanism that allows the probe holder 450 to be secured to an operating table, for example. The probe holder 450 can be utilized to hold the probe 250 at a specific height, in a static position. This configuration allows a surgical navigation system to obtain specific landmarks, such as the hip center of the patient. To calculate the hip center, the surgical team only needs to rotate the patient's leg around the hip in proximity to the probe holder 450 (with the probe 250 secured thereto) and ensure that the surgical navigation system is detecting both the fiducial marker 200 attached to the probe 250 and any additional fiducials affixed to the patient's femur.

In sum, the fiducial marker assemblies are highly beneficial because at least two of their faces can be identified by a surgical navigation system from nearly any possible orientation, thereby making it more efficient for surgical teams to perform the procedures by reducing the specificity with which the fiducial markers need to be registered with the navigation system. Further, the fiducial marker assemblies described herein can be utilized in combination with a probe and other surgical equipment that is designed to engage with the probe, thereby obviating the need for separate fiducials to be affixed to each piece of surgical equipment and allowing the procedure to be performed in a more efficient manner by obviating the need to re-register each individual piece of surgical equipment.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art, all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1-20. (canceled)

21. A fiducial marker for use with a surgical navigation system, the fiducial marker comprising:
a hexagonal prism body portion;
a triangular prism top portion;
a plurality of faces arranged on the body portion and the top portion, wherein each of the plurality of faces includes a fiducial, and wherein the plurality of faces is arranged such that at least two of the fiducials are visible to the surgical navigation system in substantially any orientation of the fiducial marker within a surgical environment; and
at least one opening on a bottom-facing side of the fiducial marker.

22. The fiducial marker of claim 21, wherein the at least one opening corresponds to a recessed interior of the fiducial marker.

23. The fiducial marker of claim 21, wherein the at least one opening includes a connector configured to engage with a probe.

24. The fiducial marker of claim 21, wherein the at least one opening includes at least a first channel configured to receive a fastener that couples the fiducial marker to patient anatomy.

25. The fiducial marker of claim 24, wherein the at least one opening includes the first channel and a second channel.

26. The fiducial marker of claim 25, wherein the first channel and the second channel are not parallel to each other.

27. The fiducial marker of claim 25, wherein the first channel and the second channel extend through the fiducial marker.

28. The fiducial marker of claim 24, wherein the first channel is threaded.

29. The fiducial marker of claim 24, further comprising a threaded aperture configured to receive a set screw to frictionally engage the fastener.

30. The fiducial marker of claim 21, wherein the bottom-facing side of the fiducial marker does not include a fiducial.

31. The fiducial marker of claim 21, wherein each of the fiducials comprises one of a passive fiducial and a QR code.

32. The fiducial marker claim 21, wherein the fiducial marker comprises a biocompatible material.

33. A probe comprising the fiducial marker of claim 21.

34. A fiducial marker for use with a surgical navigation system, the fiducial marker comprising:
a body portion;
a top portion;
a plurality of faces arranged on the body portion and the top portion, wherein each of the plurality of faces includes a fiducial, and wherein the plurality of faces is arranged such that at least two of the fiducials are visible to the surgical navigation system in substantially any orientation of the fiducial marker within a surgical environment;
first channel arranged on a bottom-facing side of the fiducial marker; and
a second channel arranged on the bottom-facing side of the fiducial marker.

35. The fiducial marker of claim 34, wherein each of the first channel and the second channel is configured to receive a respective fastener to couples the fiducial marker to patient anatomy.

36. The fiducial marker of claim 34, wherein the first channel and the second channel are not parallel to each other.

37. The fiducial marker of claim 34, wherein at least one of the first channel and the second channel is threaded.

38. The fiducial marker of claim 34, further comprising a threaded aperture configured to receive a set screw to frictionally engage a fastener inserted within at least one of the first channel and the second channel.

39. The fiducial marker of claim 34, wherein a bottom-facing side of the fiducial marker does not include a fiducial.

40. The fiducial marker of claim 34, wherein the body portion is hexagonal and the top portion is triangular.
